SEQUENCE LISTING (1 sequence)

SEQ ID NO: 1 - protein, 146 aa, Dermatophagoides pteronyssinus
MMYKILCLSLLVAAVARDQVDVKDCANHEIKKVLVPGCHGSEPCIIHRGKPFQLEAVFEANQNTKTAKIEIKASIDGLEVDVPGIDPNACHYMKCPLVKGQQYDIKYTWNVPKIAPKSENVVVTVKVMGDDGVLACAIATHAKIRD

(19) United States
(12) Patent Application Publication
(10) Pub. No.: US 2021/0000917 A1
(43) Pub. Date: Jan. 7, 2021

(54) METHODS AND COMPOSITION FOR THE TREATMENT OF ALLERGIC ASTHMA

(71) Applicants: INSERM (Institut National de la Santé et de la Recherche Médicale), Paris (FR); Université de Nantes, Nantes (FR); Centre National de la Recherche Scientifique (CNRS), Paris (FR); Institut National de Recherche Pour I'Agriculture, I'Alimentation et I'Environnement, Paris (FR)

(72) Inventors: Grégory BOUCHAUD, Nantes Cedex 3 (FR); Antoine MAGNAN, Nantes Cedex 1 (FR)

(21) Appl. No.: 16/977,883

(22) PCT Filed: Mar. 4, 2019
(86) PCT No.: PCT/EP2019/055273

§ 371 Date: Sep. 3, 2020

(30) Foreign Application Priority Data
Mar. 5, 2018 (EP) 18305231.5

(51) Int. Cl. A61K 38/17; A61P 11/00

(52) U.S. Cl. CPC A61K 38/1767; A61K 9/0019; A61P 11/00

(57) ABSTRACT

The present invention relates to a method of treating allergic asthma in a subject in need thereof comprising a step of administering to said subject a therapeutically effective amount of a polypeptide comprising or consisting of a biologically active fragment of the house-dust mite Dermatophagoides pteronyssinus Der p 2.1. Inventors have used a model allergy in a severe asthma allergic (mouse Balbc). They have surprisingly found that when they injected twice the polypeptide derp2.1 in the mouse model after a third asthma attack, the mouse presents a respiratory improvement, reduction of neutrophils and eosinophils in the broncho-alveolar lavage (BAL), an increase of regulators lymphocytes T and reduction of natural killer cells in the BAL. Thus, the polypeptide derp2.1 is a new tool to treat the allergic asthma by a restoration of lung function and reduction of inflammatory environment.

### FIELD OF THE INVENTION

The invention is in the field of allergy. More particularly, the invention relates to a polypeptide derived from a mite allergen (Der p 2) useful for the treatment of allergic asthma.

### BACKGROUND OF THE INVENTION

Allergic asthma is a chronic respiratory disease affecting 300 million people worldwide (Global Initiative for Asthma (GINA), 2011). The number of individuals with asthma has doubled during the last ten years and around 250,000 people die prematurely each year due to this condition. In the majority of cases, asthma is caused by an abnormal reactivity against some environmental antigens, also called allergens. In Western Europe, the prevalence of atopic diseases (including asthma and rhinitis) is more than 30%, thus allergic diseases are considered to be an important public health issue. Considering the pathophysiological aspects, allergic asthma is a bronchial inflammatory disease resulting from the exposition of a predisposed subject to different allergens. In Europe and the USA, the most-commonly encountered species of mite is Dermatophagoides pteronyssimus (Der p). Asthma patients are usually treated with corticosteroids, which, however, only suspend the disease and are associated with deleterious side effects. An alternative treatment for allergic asthma is based on a specific immunotherapy protocol: the repeated administration of increasing doses of allergen to induce hyposensitivity, and hence reduced symptoms when another subsequent exposure to this allergen occurs. Nevertheless, the efficacy of immunotherapy remains limited, and its efficacy is very variable between patients.

It is therefore necessary to have a treatment to treat allergic asthma and more particularly asthma exacerbations caused by allergens.

The major allergen of the house-dust mite Dermatophagoides pteronyssimus Der p 2 is a 146-amino acid protein which is further processed into a secreted mature form of 129 amino acids) after cleavage of the signal peptide (amino acids 1-17). Until now, Der p 2 has only been proposed for desensitizing a patient allergic to a house dust mite patient (e.g. a patient allergic to Dermatophagoides pteronyssinus and/or Dermatophagoides farina) but has never been shown or suggested as useful for treatment of allergic asthma in allergic or atopic patients who are asthmatic patients.

Recently, Chen et al. 2008 described that two recombinant fragments of Der p 2 (rDer p 2.1 consisting of amino acids 1 to 53 of the mature form Der p 2 and rDer p 2.2 consisting of amino acids 54 to 129) exhibited less in vivo allergenic activity and allergenicity than the Derp 2 allergen but preserved immunogenicity and may represent candidates for specific immunotherapy of house-dust mite allergy.

However, the effects of the Derp 2 allergen or derivatives thereof such as rDer p 2.1 and rDer p 2.2 on the respiratory function have never been studied. Indeed, only the induction of IgE responses has been studied after immunization. Moreover, the action of the derivatives rDer p 2.1 and rDer p 2.2 has never been studied in vivo in a severe asthma allergic mouse model.

### SUMMARY OF THE INVENTION

The invention relates to a method of treating allergic asthma in a subject in need thereof comprising a step of administering to said subject a therapeutically effective amount of a polypeptide comprising or consisting of a biologically active fragment of the house-dust mite Dermatophagoides pteronys sinus Der p 2. In particular, the invention is defined by the claims.

### DETAILED DESCRIPTION OF THE INVENTION

In the present invention, inventors have used a model allergy in a severe asthma allergic (mouse Balbc). They have surprisingly found that when they injected twice the polypeptide derp2.1 in the mouse model after a third asthma attack, the mouse presents a respiratory improvement, reduction of neutrophils and eosinophils in the broncho-alveolar lavage (BAL), an increase of regulators lymphocytes T and reduction of natural killer cells in the BAL. Thus, the polypeptide derp2.1 is a new tool to treat the allergic asthma by a restoration of lung function and reduction of inflammatory environment.

Accordingly, the invention relates to a method of treating allergic asthma in a subject in need thereof comprising a step of administering to said subject a therapeutically effective amount of a polypeptide comprising or consisting of a biologically active fragment of the house-dust mite Dermatophagoides pteronyssinus Der p 2.

In the context of the invention, the term “treatment” or “treat” as used herein, refers to a treatment as well as curative or disease modifying treatment, including treatment of subject at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “allergic asthma” is the most common form of asthma. The allergic asthma is triggered by inhaling allergens. As used herein, the term “asthma” refers to a common long-term inflammatory disease of the airways of the lungs. Symptoms of asthma include coughing, shortness of breath, and chest tightness. As used herein, the term “allergen” refers to a substance that can cause an allergic reaction. Typically, the allergen triggers an immune response which is called as an allergic reaction. Allergens are well-known to the skilled in the art. Common environmental allergens which induce allergic diseases are found in pollen (e.g. tree, herb, weed and grass pollen allergens), food, animal danders, hair and/or saliva (from e.g. dog, cat, horse, rat, mouse etc.), molds, fungal spores and venoms (e.g. insect or batracian venom). In the context of the invention, the allergen is acari. Acari, also called as acarina are a class of arachnids containing mites and ticks. Typically, in the context of the invention, the allergen is an acari, Dermatophagoides pteronyssinus which is considered as house dust mite.

Another type of allergic reaction is an atopy reaction. Atopy is a genetic predisposition of an individual to produce high quantities of IgE in response to allergens in the environment (pollens, house dust mites, moulds, cat dander, foods etc). Atopy can have a hereditary component, although contact with the allergens should occur before the hypersensitivity reaction may develop. In another embodiment, the allergic asthma is a severe allergic. As used herein, the term “severe allergic asthma” refers to acute asthma attacks and chronic worsening of inflammation being allergens inducing an immune, IgE mediated response.

As used herein, the term “subject” refers to any mammals, such as a rodent, a feline, a canine, and a primate. Particularly, in the present invention, the subject is a human. In a particular embodiment, the subject is a human who suffers or is susceptible to have allergic asthma. In a particular embodiment, the subject has allergic asthma to acari. In another particular embodiment, the subject suffers or is susceptible to suffer from severe asthma allergic.

As used herein, the term “polypeptide” refers both short peptides with a length of at least two amino acid residues and at most 10 amino acid residues, oligopeptides (11-100 amino acid residues), and longer peptides (the usual interpretation of “polypeptide”, i.e. more than 100 amino acid residues in length) as well as proteins (the functional entity comprising at least one peptide, oligopeptide, or polypeptide which may be chemically modified by being glycosylated, by being lipidated, or by comprising prosthetic groups). The polypeptides of the invention may be produced by any suitable means, as will be apparent to those of skill in the art. In order to produce sufficient amounts of polypeptides for use in accordance with the present invention, expression may conveniently be achieved by culturing under appropriate conditions recombinant host cells containing the polypeptide of the invention. In particular, the polypeptide is produced by recombinant means, by expression from an encoding nucleic acid molecule. Systems for cloning and expression of a polypeptide in a variety of different host cells are well known. When expressed in recombinant form, the polypeptide is in particular generated by expression from an encoding nucleic acid in a host cell. Any host cell may be used, depending upon the individual requirements of a particular system. Suitable host cells include bacteria mammalian cells, plant cells, yeast and baculovirus systems. Mammalian cell lines available in the art for expression of a heterologous polypeptide include Chinese hamster ovary cells. HeLa cells, baby hamster kidney cells and many others. Bacteria are also preferred hosts for the production of recombinant protein, due to the ease with which bacteria may be manipulated and grown. A common, preferred bacterial host is E coli. The polypeptides of the invention and fragments thereof according to the invention can exhibit post-translational modifications, including, but not limited to glycosylations, (e.g., N-linked or O-linked glycosylations), myristylations, palmitylations, acetylations and phosphorylations (e.g., serine/threonine or tyrosine). In some embodiments, it is contemplated that polypeptides used in the therapeutic methods of the present invention may be modified in order to improve their therapeutic efficacy. Such modification of therapeutic compounds may be used to decrease toxicity, increase circulatory time, or modify biodistribution. For example, the toxicity of potentially important therapeutic compounds can be decreased significantly by combination with a variety of drug carrier vehicles that modify biodistribution. In example adding dipeptides can improve the penetration of a circulating agent in the eye through the blood retinal barrier by using endogenous transporters.

In the context of the invention, the polypeptide comprises or consists of a biologically active fragment of the house-dust mite Dermatophagoides pteronyssinus Der p 2.

As used herein, the term “Der p 2” refers to the major allergen of the house-dust mite (HDM) Dermatophagoides pteronyssimus which is recognized by approximatively 90% of mite allergic patients. The naturally occurring Der p 2 consists of the amino acid sequence consisting of amino acids 18 to 146 of SEQ ID NO: 1. Thus, SEQ ID NO: 1 corresponds to the sequence of Der p 2 + the signal peptide that is referenced with the UniProtKB/Swiss-Prot Accession number P49278. As used herein, the term “Der p 2.1” refers to the polypeptide having a sequence consisting of amino acids 18 to 70 of SEQ ID NO: 1. As used, the term “Der p 2.2” refers to the polypeptide consisting of the amino acid sequence 71 to 146 of SEQ ID NO: 1. Typically, the invention relates to an isolated polypeptide comprising a biologically active fragment of the allergen of the house-dust mite Dermatophagoides pteronyssinus Der p 2, wherein said fragment comprises:
a) the sequence consisting of amino acids 18 to 70 of SEQ ID NO: 1;
b) a sequence at least 80% identical to the sequence of (a);
c) at least six consecutive amino acids of the sequence of (a) or (b); for use in the treatment of allergic asthma in a patient in need thereof.

The sequence of the polypeptide of the invention is represented in Table A below:

TABLE A
Amino acid sequence of the polypeptide
of the invention.

| SEQ | | Nomenclature |
|---|---|---|
| ID | | used in the |
| number | Sequence | patent application |
| Poly- | MMYKILCLSLLVAA | Mite group 2 allergen |
| peptide | VARDQVDVKDCANH | Der p 2 (Complete form |
| SEQ ID | EIKKVLVPGCHGSE | before processing into |
| NO: 1 | PCIIHRGKPFQLEA | a mature form by |
| | VFEANQNTKTAKIE | cleavage of the signal |
| | IKASIDGLEVDVPG | peptide) |
| | IDPNACHYMKCPLV | |
| | KGQQYDIKYTWNVP | |
| | KIAPKSENVVVTVK | |
| | VMGDDGVLACAIAT | |
| | HAKIRD | |

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% (5 of 100) of the amino acid residues in the subject sequence may be inserted, deleted, or substituted with another amino acid.

In the frame of the present application, the percentage of identity is calculated using a global alignment (i.e., the two sequences are compared over their entire length). Methods for comparing the identity and homology of two or more sequences are well known in the art. The «needle» program, which uses the Needleman-Wunsch global alignment algorithm (Needleman and Wunsch, 1970 J. Mol. Biol. 48:443-453) to find the optimum alignment (including gaps) of two sequences when considering their entire length, may for example be used. The needle program is for example available on the ebi.ac.uk world wide web site. The percentage of identity in accordance with the invention is preferably calculated using the EMBOSS::needle (global) program with a “Gap Open” parameter equal to 10.0, a “Gap Extend” parameter equal to 0.5, and a Blosum62 matrix.

Polypeptides consisting of an amino acid sequence “at least 80%, 85%, 90%, 95%, 96%, 97%, 98% or 99% identical” to a reference sequence may comprise mutations such as deletions, insertions and/or substitutions compared to the reference sequence. The polypeptide consisting of an amino acid sequence at least 80%, 85%, 90%, 95%, 96%, 97%, 98% or 99% identical to a reference sequence may correspond to an allelic variant of the reference sequence. It may for example only comprise substitutions compared to the reference sequence. The substitutions preferably correspond to conservative substitutions as indicated in the table below.

| Conservative substitutions | Type of Amino Acid |
|---|---|
| Ala, Val, Leu, Ile, Met, Pro, | Amino acids with aliphatic hydrophobic |
| Phe, Trp | side chains |
| Ser, Tyr, Asn, Gln, Cys | Amino acids with uncharged but polar |
| | side chains |
| Asp, Glu | Amino acids with acidic side chains |
| Lys, Arg, His | Amino acids with basic side chains |
| Gly | Neutral side chain |

In one particular embodiment, the isolated polypeptide for use in the treatment of allergic asthma in a patient in need thereof, comprises a biologically active fragment of at most 100 consecutive amino acids of the allergen of the house-dust mite Dermatophagoides pteronys sinus Der p 2, wherein said fragment comprises:
a) the sequence consisting of amino acids 18 to 70 of SEQ ID NO: 1; or
b) a sequence at least 80% identical to the sequence of (a); or
c) at least six consecutive amino acids of the sequence of (a) or (b).

The biologically active fragment of the house-dust mite Dermatophagoides pteronys sinus Der p 2 consist in:
a) the sequence consisting of amino acids 18 to 70 of SEQ ID NO: 1; or
b) a sequence having at least 80% of identity with the sequence of (a); or
c) at least six consecutive amino acids of the sequence of (a) or (b).

The polypeptides according to the invention, for use in the treatment of allergic asthma as further described herein, encompass polypeptides comprising or consisting of fragments of Der p 2 allergen, provided the fragments are biologically active.

In the frame of the invention, the biologically active fragment may for example comprise at least 6, 8, 10, 12, 15, 18, 20, 25, 30, 35, 40, 45, 50, 55, 60, 65, 70, 75, 80, 85, 90, 95 or 100 consecutive amino acids of Der p 2. According to the invention, a polypeptide comprising a biologically active fragment of at most 100 consecutive amino acids of Der p 2 does not contain more than 100 consecutive amino acids of Der p 2. Indeed, “a polypeptide comprising a biologically active fragment of at most 100 consecutive amino acids of Der p 2” according to the invention refers to a polypeptide of at most 100 consecutive amino acids of Der p 2. Accordingly, said “polypeptide comprising or consisting of a biologically active fragment of Der p 2” is not the full length mature Der p 2 protein.

As used herein, a “biologically active” fragment refers to a fragment exhibiting at least one, preferably all, of the biological activities of Derp 2.1, provided the biologically active fragment retains the capacity of decreasing the airway hyper responsiveness and/or asthma exacerbations. The biologically active fragment may for example be characterized in that it is capable of improving respiratory function when assessed by plethysmography and/or decreasing pulmonary inflammation (e.g. decrease of the number of inflammatory cells in lung or in BALF (broncho-alveolar lavage fluid) (when assessed through lund cytology or BALF cytology).

The skilled in the art can easily determine whether a polypeptide fragment of Der p 2 is biologically active. To check whether the newly generated polypeptides decrease the airway hyper responsiveness in the same way than the initially characterized polypeptide rDer p 2.1 a lung function measurement and broncho-alveolar lavage (BAL) cytology in a mouse model of Der p-induced allergy mimicking asthma and allergen-driven exacerbation (such as described in Example) may be performed with each polypeptide. Additionally, a time-course and a dose-response performed in said mouse model of Der p-induced allergy mimicking asthma and allergen-driven exacerbation will determine the optimal conditions for each polypeptide.

The inventors have identified that only the fragment of Derp 2.1 (unlike to Der p 2.2) both known as exhibiting less in vivo allergenic activity and allergenicity than the full-length Derp 2 allergen but preserving immunogenicity enables to decrease airway hyperresponsiveness and prevent asthma exacerbations (by decreasing the pulmonary inflammation via a decrease of the number of inflammatory cells, in particular Th2-cytokine-producing cells involved in allergic asthma (e.g. eosinophils) and a decrease of the level of inflammatory cytokines (e.g.).

Therefore, the present invention provides an isolated polypeptide comprising or consisting of a biologically active fragment of Der p 2, wherein said fragment comprises or consists of:
a) the sequence consisting of amino acids 18 to 70 of SEQ ID NO: 1;
b) a sequence at least 80, 85, 90, 95, 96, 97, 98 or 99% identical to the sequence of (a);
c) at least 6, 8, 10, 12, 15, 18, 20, 25, 30, 35, 40, 45, 50, 55, 60, 65, 70, 75, 80 or 85, 90, 95 or 100 consecutive amino acids of the sequence of (a) or (b); for use in the treatment of allergic asthma in a patient in need thereof.

In a particular embodiment, the polypeptide for use consists of:
a) the sequence consisting of amino acids 18 to 70 of SEQ ID NO: 1;
b) a sequence at least 80, 85, 90, 95, 96, 97, 98 or 99% identical to the sequence of (a);
c) at least 6, 8, 10, 12, 15, 18, 20, 25, 30, 35, 40, 45, 50 consecutive amino acids of the sequence of (a) or (b).

By an “isolated” polypeptide, it is intended that the polypeptide is not present within a living organism, e.g. within a house-dust mite such as Dermatophagoides pteronyssimus.

The isolated polypeptide is particularly purified. The term “purified” employed herein means that the polypeptide of the invention (e.g. Der p 2.1) contains less than 5% other components from the host namely (poly) peptides contaminants.

The polypeptides of the invention may be produced by any method well known in the art (e.g. chemical synthesis or recombinant techniques).

Examples of chemical synthesis technologies are solid phase synthesis and liquid phase synthesis. As a solid phase synthesis, for example, the amino acid corresponding to the C-terminus of the polypeptide to be synthesized is bound to a support which is insoluble in organic solvents, and by alternate repetition of reactions, one wherein amino acids with their amino groups and side chain functional groups protected with appropriate protective groups are condensed one by one in order from the C-terminus to the N-terminus, and one where the amino acids bound to the resin or the protective group of the amino groups of the polypeptides are released, the polypeptide chain is thus extended in this manner. Solid phase synthesis methods are largely classified by the tBoc method and the Fmoc method, depending on the type of protective group used. Typically used protective groups include tBoc (t-butoxycarbonyl), Cl—Z (2-chlorobenzyloxycarbonyl), Br—Z (2-bromobenzyloyycarbonyl), Bzl (benzyl), Fmoc (9-fluorenylmcthoxycarbonyl), Mbh (4,4′-dimethoxydibenzhydryl), Mtr (4-methoxy-2, 3, 6-trimethylbenzenesulphonyl), Trt (trityl), Tos (tosyl), Z (benzyloxycarbonyl) and Clz—Bzl (2, 6-dichlorobenzyl) for the amino groups; NO2 (nitro) and Pmc (2,2,5,7,8-pentamethylchromane-6-sulphonyl) for the guanidino groups); and tBu (t-butyl) for the hydroxyl groups). After synthesis of the desired peptide, it is subjected to the de-protection reaction and cut out from the solid support. Such peptide cutting reaction may be carried with hydrogen fluoride or tri-fluoromethane sulfonic acid for the Boc method, and with TFA for the Fmoc method.

Alternatively, the polypeptide may be synthesized using recombinant techniques. In this case, a nucleic acid encoding a polypeptide according to the invention (further referred to as “a nucleic acid according to the invention”) is cloned into an expression vector. The nucleic acid of the invention is preferably placed under the control of expression signals (e.g. a promoter, a terminator and/or an enhancer) allowing its expression. The expression vector is then transfected into a host cell (e.g. a human, CHO, mouse, monkey, fungal or bacterial host cell), and the transfected host cell is cultivated under conditions suitable for the expression of the polypeptide. For instance, in the section “Examples”, the term “rDer p 2.1” designates the recombinant Der p 2.1 polypeptide expressed by Escherichia coli (E. coli). The term “expression vector” is meant to include any type of genetic construct containing a nucleic acid coding for a gene product in which part or all of the nucleic acid encoding sequence is capable of being transcribed.

The method of producing the polypeptides of the invention may optionally comprise the steps of purifying said polypeptide, chemically modifying said polypeptide, and/or formulating said polypeptide into a pharmaceutical composition.

In one embodiment, the polypeptides of the invention may comprise a tag. A tag is an epitope-containing sequence which can be useful for the purification of the polypeptides. It is attached to by a variety of techniques such as affinity chromatography, for the localization of said peptide or polypeptide within a cell or a tissue sample using immunolabeling techniques, the detection of said peptide or polypeptide by immunoblotting etc. Examples of tags commonly employed in the art are the GST (glutathion-S-transferase)-tag, the FLAG™-tag, the Strep-tag™, V5 tag, myc tag, His tag (which typically consists of six histidine residues), etc.

In another embodiment, the polypeptides of the invention may comprise chemical modifications improving their stability and/or their biodisponibility. Such chemical modifications aim at obtaining polypeptides with increased protection of the polypeptides against enzymatic degradation in vivo, and/or increased capacity to cross membrane barriers, thus increasing its half-life and maintaining or improving its biological activity. Any chemical modification known in the art can be employed according to the present invention. Such chemical modifications include but are not limited to:
replacement(s) of an amino acid with a modified and/or unusual amino acid, e.g. a replacement of an amino acid with an unusual amino acid like Nle, Nva or Orn; and/or
modifications to the N-terminal and/or C-terminal ends of the peptides such as e.g. N-terminal acylation (preferably acetylation) or desamination, or modification of the C-terminal carboxyl group into an amide or an alcohol group;
modifications at the amide bond between two amino acids: acylation (preferably acetylation) or alkylation (preferably methylation) at the nitrogen atom or the alpha carbon of the amide bond linking two amino acids;
modifications at the alpha carbon of the amide bond linking two amino acids such as e.g. acylation (preferably acetylation) or alkylation (preferably methylation) at the alpha carbon of the amide bond linking two amino acids.
chirality changes such as e.g. replacement of one or more naturally occurring amino acids (L enantiomer) with the corresponding D-enantiomers;
retro-inversions in which one or more naturally-occurring amino acids (L-enantiomer) are replaced with the corresponding D-enantiomers, together with an inversion of the amino acid chain (from the C-terminal end to the N-terminal end);
azapeptides, in which one or more alpha carbons are replaced with nitrogen atoms; and/or
betapeptides, in which the amino group of one or more amino acid is bonded to the β carbon rather than the a carbon.

Another strategy for improving biological activity is the utilization of water-soluble polymers. Various water-soluble polymers have been shown to modify biodistribution, improve the mode of cellular uptake, change the permeability through physiological barriers; and modify the rate of clearance from the body. To achieve either a targeting or sustained-release effect, water-soluble polymers have been synthesized that contain drug moieties as terminal groups, as part of the backbone, or as pendent groups on the polymer chain.

Polyethylene glycol (PEG) has been widely used as a drug carrier, given its high degree of biocompatibility and ease of modification. Attachment to various drugs, proteins, and liposomes has been shown to improve residence time and decrease toxicity. PEG can be coupled to active agents through the hydroxyl groups at the ends of the chain and via other chemical methods; however, PEG itself is limited to at most two active agents per molecule. In a different approach, copolymers of PEG and amino acids were explored as novel biomaterials which would retain the biocompatibility properties of PEG, but which would have the added advantage of numerous attachment points per molecule (providing greater drug loading), and which could be synthetically designed to suit a variety of applications.

In a second aspect, the invention also provides a method for treating allergic asthma in a subject in need thereof comprising a step of administering to said subject a therapeutically effective amount of a nucleic acid encoding and/or vectors expressing a polypeptide comprising or consisting of a biologically active fragment of Der p 2 as described above.

Such nucleic acids or vectors find use when a polypeptide comprising or consisting of a biologically active fragment of Der p 2 is intended to be administered to a patient in the frame of a gene therapy. In this case, the nucleic acid is preferably present on an expression vector, on which the sequence coding for the polypeptide is placed under the control of expression signals (e.g. a promoter, a terminator and/or an enhancer) allowing its expression. The vector may for example correspond to a viral vector such as an adenoviral or a lentiviral vector.

Alternatively, a synthetic vector may be used in combination with nucleic acids encoding a polypeptide comprising or consisting of a biologically active fragment of Der p 2.

Such synthetic vector may be specific chemical molecules may be tetrafunctional block copolymers which have been shown able to safely increase the transfection efficiency of reporter or therapeutic genes in lung. This class of vector has been proposed for intracellular delivery of nucleic acids (WO 03/066104).

Accordingly, a tetrafunctional copolymer suitable for the invention may be a compound of formula (I):

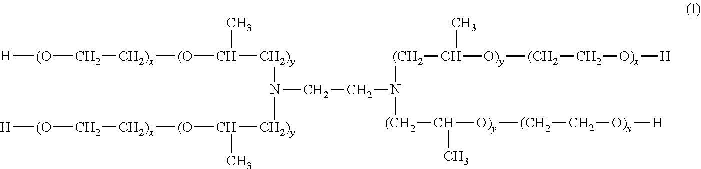

wherein x and y represents, independently of one another, an integer of between 1 and 500 with x having a value such that said molecule comprises at least 40% by weight of ethylene oxide units.

The invention may also be implemented with derivatives of compounds of formula (I).

As used herein, the term “derivative” is intended to cover compounds which have the chemical structure of general formula I but which also carry secondary chemical or biological functions or entities capable of conferring on them complementary properties. Particularly representative of these derivatives are compounds of general formula (I) in which there is also as least one intra- or extracellular targeting unit. By way of non-limiting illustration of these targeting units, mention may more particularly be made of peptides carrying a nuclear localization sequence, or peptides which recognize receptors present at the surface of certain cells.

A compound of general formula (I) has no more than 85% by weight of ethylene oxide units.

A compound of general formula (I) has in particular approximately between 40 and 80% by weight of ethylene oxide units.

According to a preferred variant of the invention, the molecules of compounds of general formula (I) also have a molecular weight of at least 800 g/mol, and more preferably of between 1000 and 25 000 g/mol.

According to a preferred embodiment of the invention, the compounds of general formula have an EO/PO unit ratio of between 0.5 and 1.5, and preferably of the order of 1±0.2.

As compounds of general formula (I) that are most particularly suitable for the present invention, mention may more particularly be made of molecules having, respectively, a molecular weight of 1650 g for an EO/PO ratio of 15:16 (for example poloxamine 304), of 5500 g/mol for an EO/PO ratio of 50:56 (for example poloxamine 704) and of 6700 g/mol for an EO/PO ratio of 61:68 (for example poloxamine 904).

According to a preferred embodiment a compound of formula (I) may be selected in the group consisting of poloxamine 304, 704, 904, and mixture thereof.

According to a preferred embodiment, the composition is free of sodium phosphate and/or of glucose.

A tetrafunctional copolymer suitable for the invention is used in a cationic form.

A compound of general formula (I) is preferably used in the form of one of its salts, and more preferably in a cationic form. To do this, the composition claimed combines with said compound a preferably mineral salt, and more preferably an alkali metal salt or an alkaline-earth metal salt. It may in particular be chosen from sodium chloride, potassium chloride or lithium chloride and sodium thiocyanate, or more preferably calcium chloride (CaCl₂) or magnesium chloride (MgCl₂).

This salt may be introduced in isotonic, hypotonic or hypertonic amount.

The inventors have also established the advantage of controlling the pH and/or the ionic composition of the formulation, in order to be sure that the copolymer of formula (I) is in its cationic form.

A pH of 6.5 to 8, preferably 7 to 7.8, more preferably 7.4, proves to be particularly advantageous.

According to another embodiment of the invention, the composition is formulated in a medium referred to as Tyrode's (medium containing 3 mM CaCl₂, 2 mM MgCl₂, 6 mM KCl, 140 mM NaCl, 10 mM glucose and 10 mM Hepes, pH 7.4) (Tyrode Pharmacology. Philadelphia, 1908, 2nd edition, 1912). The presence of the Tyrode's makes it possible in particular to control the ionic composition of the formulation and the pH and, consequently, the use of the compound of formula (I) in a cationic form.

The preparation of a nucleic acid molecule with a poloxamine in a salt medium, and in particular in presence of Tyrode medium may be made as described in WO 03/066104.

Another aspect of the invention relates to a method for treating allergic asthma comprising administering to a subject in need thereof an effective amount of a nucleic acid encoding and/or a vector expressing a polypeptide according to the invention as described above.

In another embodiment, the polypeptide as described above can be modified to be inserted in the particles. The particles are selected from the group consisting of but not limited to natural vesicles such as exosomes, liposomes or synthetic vesicles such as nanoparticles. In a particular embodiment, the polypeptide according to the invention is inserted in synthetic vesicles such as nanoparticles.

Polymeric micro/nanoparticles refer to micron to nano sized drug particles coated with layer(s) of polymer(s) and/or other materials. A polymer is a large molecule, or macromolecule, composed of many repeated subunits. The molecular weight can range from 500 to >100,000 Daltons. A biodegradable polymer defined in the biodegradable micro/nanoparticles section is preferred for use in this invention. The Z-average mean diameter of the polymeric particles of this invention range from 100 micron to below 100 nm, preferably from 50 micron to 10 micron, more preferably from 10 micron to 2 micron, still more preferably from 2 micron to 500 nm, even more preferably from 500 nm to 100 nm, and most preferably below 100 nm. Biodegradable polymeric nanoparticles where the drug is coated by polymeric materials are deemed to be very efficient drug delivery systems. It should be highlighted that the liberation of the polymer encapsulated drug can be carefully controlled by total surface area or the particle size, or the coating materials; and the drug concentration in the target site is maintained within the therapeutic window. Biodegradable polymers are considered as ideal biomaterials for the development of controlled- and sustained-release drug delivery systems as well as therapeutic devices.

A liposome is a spherical vesicle having at least one lipid bilayer, which fall in the category of microparticles or nanoparticles. Liposomes can be prepared by disrupting biological membranes (such as by sonication). Liposomes are most often composed of phospholipids, especially phosphatidylcholine, but may also include other lipids, such as egg phosphatidylethanolamine, so long as they are compatible with lipid bilayer structure. Lipid complexation with drug and other materials is also regarded as liposome in this invention. A liposome design may employ surface ligands for attaching to unhealthy tissue. The polypeptide could be incorporated into the liposome in either hydrophilic or hydrophobic region or both. The major types of liposomes are the multilamellar vesicles (MLV, with several lamellar phase lipid bilayers), the small unilamellar liposome vesicles (SUV, with one lipid bilayer), the large unilamellar vesicles (LUV), and the cochleate vesicles. A less desirable form are multivesicular liposomes in which one vesicle contains one or more smaller vesicles.

In another embodiment, the polypeptide according to the invention is inserted in bacterium-like particles. Production of bacterium-like particles is well-known in the art. Typically, the bacterium (e.g Lactococcus lactis) is killed at low pH and high temperature, which generates bacterium-like particles (BLPs) that are non-living and deprived of intact surface proteins and intracellular content. The BLPs activate antigen presenting cells of the innate immune system by Toll-like receptor 2 (TLR-2) interaction (Ramirez K,et al 2010). The technology to obtain BLP is described in the review Van Braeckel-Budimir et al 2013.

As used herein the terms “administering” or “administration” refer to the act of injecting or otherwise physically delivering a substance as it exists outside the body (e.g. polypeptide derp2.1) into the subject, such as by mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. In the context of the invention, the subject is administered with the polypeptide of the invention by topical administration. The topical administration is performed by a patch application. In a further embodiment, the subject is administered with the polypeptide of the invention by subcutaneous, nebulization or sublingual administration. In another embodiment, the subject is administered with the polypeptide of the invention by oral administration. The oral administration is performed by tablets, gel capsules, powders, granules and oral suspensions or solutions, sublingual and buccal administration forms.

A “therapeutically effective amount” is intended for a minimal amount of active agent which is necessary to impart therapeutic benefit to a subject. For example, a “therapeutically effective amount” to a subject is such an amount which induces, ameliorates or otherwise causes an improvement in the pathological symptoms, disease progression or physiological conditions associated with or resistance to succumbing to a disorder. It will be understood that the total daily usage of the compounds of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidential with the specific compound employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The present invention relates also to a pharmaceutical composition comprising the polypeptide as described above which is inserted or not in a BLP. The polypeptide may be combined with pharmaceutically acceptable excipients, and optionally sustained-release matrices, such as biodegradable polymers, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to one or more of the following agents: solvents such as olive oil, olive oil refined, cottonseed oil, sesame oil, sunflower seed oil, peanut oil, wheat germ oil, soybean oil, jojoba oil, evening primrose oil, coconut oil, palm oil, sweet almond oil, aloe oil, apricot kernel oil, avocado oil, borage oil, hemp seed oil, macadamia nut oil, rose hip oil, pecan oil, hazelnut oil, sasanqua oil, rice bran oil, shea butter, corn oil, camellia oil, grape seed oil, canola oil, castor oil, and combinations thereof, preferably olive oil refined, emulsifiers, suspending agents, decomposers, binding agents, excipients, stabilizing agents, chelating agents, diluents, gelling agents, thickening agent such as beeswax and/or petroleum jelly, preservatives, lubricants, absorption delaying agents, liposomes, antioxidants such as butylhydroxytoluene or butylhydroxyanisole, and the like. The pharmaceutical compositions of the present invention for topical, oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms. Particularly, the pharmaceutical composition is formulated into a topical formulation that can be directly applied to the skin. The topical formulation suitable for the pharmaceutical composition may be an emulsion, a gel, an ointment, a cream, a patch, an embrocation, an aerosol, a spray, a lotion, a serum, a paste, a foam, or a drop. In the context of the invention, the pharmaceutical composition is a patch. In one embodiment of this application, the pharmaceutical composition is formulated into an external preparation by admixing the extract according to this application with a base such as those that are well known and commonly used in the art.

In one embodiment, the polypeptide of the invention is administered in combination with a classical treatment of allergic asthma

Thus, the invention also refers to a method of treating allergic asthma in a subject in need thereof comprising administering to said subject a therapeutically effective amount of a polypeptide comprising or consisting of a biologically active fragment of the house-dust mite Dermatophagoides pteronyssinus Der p 2 in combination with a classical treatment of allergic asthma.

As used herein, the term “classical treatment” refers to any compound, natural or synthetic, used for the treatment of an allergic asthma.

According to the invention, compound used for the treatment of an allergic asthma may be selected in the group consisting in: corticosteroids such as flunisolide, ciclesonide, mometasone, fluticasone, budesonide, beclomethasone and prednisome; bronchodilators such as epinephrine, racepinephrine, indacaterol, albuterol, levalbuterol, olodaterol, formoterol, arformoterol, pirbuterol, terbutaline, metaproterenol, salmeterol, ipratropium, aclidinium, tiotropium, umeclidinium, glycopyrrolate and revefenacin; methylxanthines such as theophylline, dyphylline, and aminophylline; anti-leukotriene drugs such as montelukast, zafirkulast and zileuton; interleukin inhibitors such as reslizumab, dupilumab, benralizumab, mepolizumab; mast cell stabilizer such as cromolyn; allergen immunotherapy such as grastek, oralair and ragwitek; and omalizumab.

Accordingly, the invention relates to i) a polypeptide comprising or consisting of a biologically active fragment of the house-dust mite Dermatophagoides pteronyssinus Der p 2 and ii) a classical treatment as a combined preparation for use in treating a subject suffering from allergic asthma.

In a particular embodiment, i) the polypeptide comprising or consisting of a biologically active fragment of the house-dust mite Dermatophagoides pteronys sinus Der p 2 and ii) a classical treatment as a combined preparation for simultaneous, separate or sequential use in treating a subject suffering from allergic asthma.

As used herein, the terms “combined treatment”, “combined therapy” or “therapy combination” refer to a treatment that uses more than one medication. The combined therapy may be dual therapy or bi-therapy.

As used herein, the term “administration simultaneously” refers to administration of 2 active ingredients by the same route and at the same time or at substantially the same time. The term “administration separately” refers to an administration of 2 active ingredients at the same time or at substantially the same time by different routes. The term “administration sequentially” refers to an administration of 2 active ingredients at different times, the administration route being identical or different.

As used herein the terms “administering” or “administration” refer to the act of injecting or otherwise physically delivering a substance as it exists outside the body (e.g., an inhibitor of IRE1α) into the subject, such as by mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

The invention will be further illustrated by the following figures and examples. However, these examples and figures should not be interpreted in any way as limiting the scope of the present invention.

### FIGURES

FIG. 1: Mouse model of HDM-induced asthma and Derp 2.1 therapeutic potential on hyper responsiveness BAL and lung infiltrate. A, Schematic representation of the HDM allergic model. B and C, Measurement of airway resistances and elastance to increasing doses of methacholine in CTL (white circles), asthmatic-like (HDM; black circles) and treated mice (Derp2.1; black squares) on day 38 (n=7-9 mice/group). D, Total cells, lymphocytes (LT), macrophages (MΦ), neutrophils (PNN) and eosinophils (PNEo) counts in BAL (day 38) in CTL (white bars), asthmatic-like (HDM; black bars) and treated mice (Derp 2.1; black squares bars). E, Serum level of Der p 2-specific IgE in CTL (white circles), asthmatic-like (HDM; black circles) and treated mice (Derp2.1; black squares). F, Frequency of lung Treg cells (n=4/7 mice/group). G, Number of lung natural killer cells (n=2 mice/group) in CTL (black circles), asthmatic-like (RA; white squares) and treated mice (Derp 2.1; white triangle). Data are represented as mean±SEM. *p<0.05, **p<0.01, ***p<0.001, ****p<0.0001

### EXAMPLE

### Material & Methods

### Animal Procedures

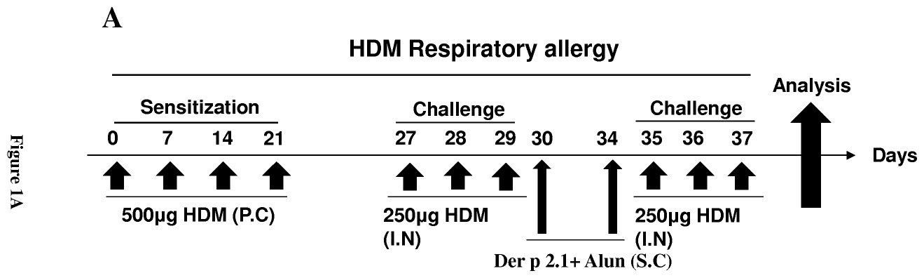

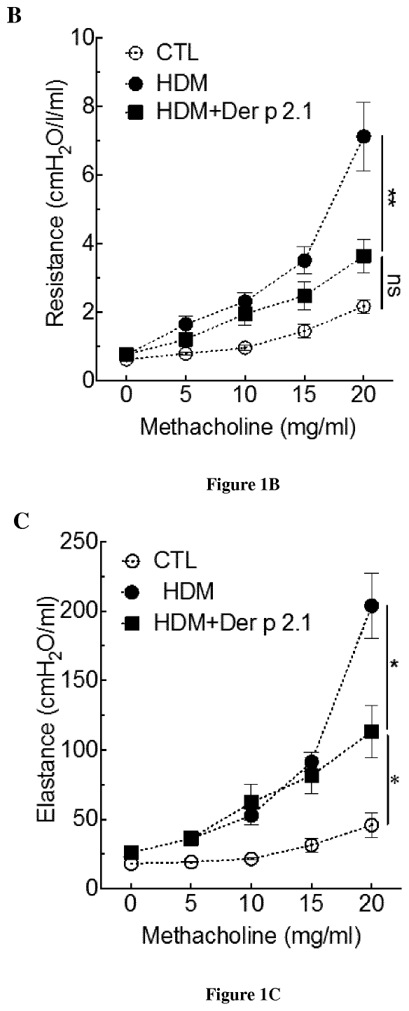

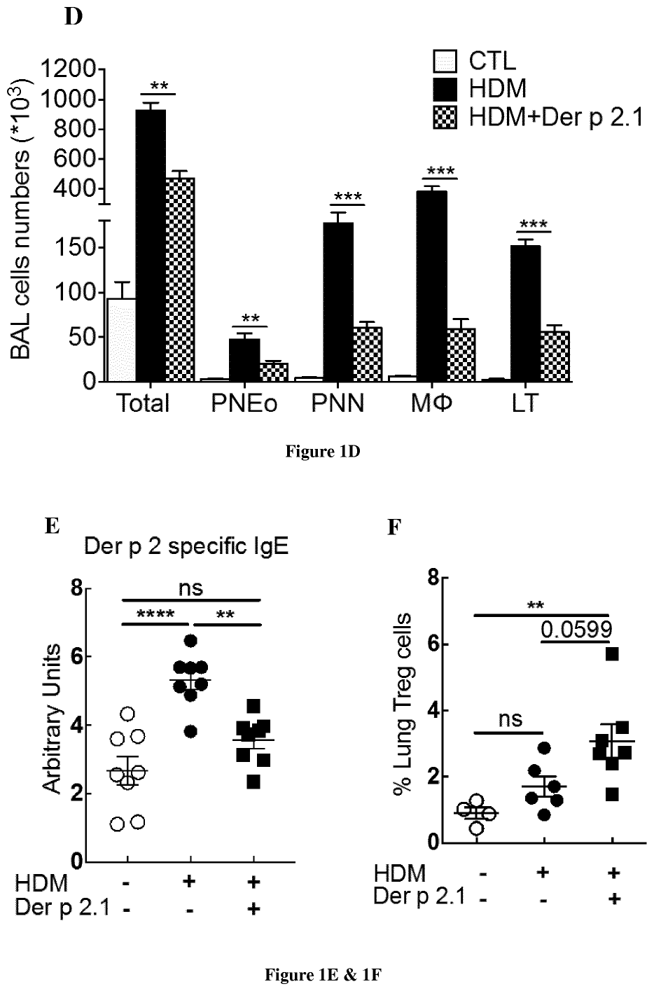

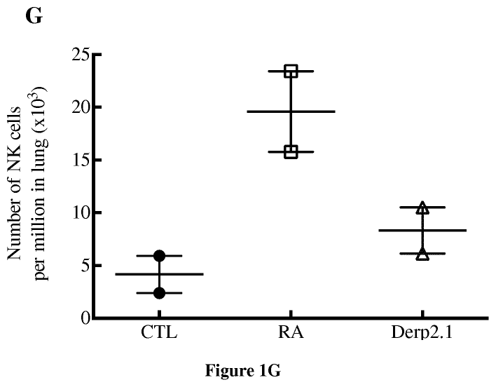

BALB/c female mice aged 6 to 8 weeks were purchased from Charles River Breeding Laboratories and used for all experiments. The animals were housed in UTE IRS-UN platform (Nantes, France). The mice were maintained under specific pathogen-free, temperature-controlled conditions under a strict 12-hour light-dark cycle and were given free access to food and water. The mice were sensitized on days 0, 7, 14 and 21 by percutaneous application of 500 μg of total extract of Dermatophagoïdes farinae (HDM) (Stallergenes, Antony, France) in 20 μl of dimethylsulfoxide (Sigma- Aldrich, St Louis, Mo.) on the ears without any synthetic adjuvant. The mice were challenged intranasally with 250 μg of HDM (House Dust Mite) in 40 μl of sterile PBS on day 27, 28 and 29 to induce asthma and again on days 35, 36 and 37 to induce asthma exacerbation. Animals were anesthetized with 100 μg of xylazine (ROMPUN 2%, Bayer, Lyon, France, 15 mg/kg) intraperitoneally and 100 μl of ketamine (IMALGENE100, Merial, Lyon, France, 80 mg/kg) for both sensitizations and challenges. Mice were sacrified by sublethal injection of dolethal (vetoquinol) on day 39 (FIG. 1A). For dynamic lung resistance measurements, mice were anesthetized with 200 μl of a ketamine-xylazine mix and paralyzed with 100 μl of rocuronium bromide (ESMERON, Organon, Kloosterstraat, The Netherlands, 10 mg/ml) intraperitoneally.

### Derivative Peptide Injection

Derivative peptide (Derp 2.1) was purified as previously described by Chen et al. 1Peptide was injected on day 30 after de third challenge and on day 34 before the fourth challenge. Derp 2.1 was solubilized with sterile PBS to a final concentration of 100 μg/ml. pproximately 200 μl of a solution of Alum (Imject Alum, Thermo Scientific) containing 20 μg of the peptide was injected subcutaneously in the neck of the mice.

### BAL Preparation

One milliliter of sterile PBS was administered to mice through a flexible catheter. Cells and supernatants from the removed fluid were separated by centrifugation. The total cell number was determined by using flow cytometry (see below). Supernatants were stored at −20° C.

### Flow Cytometry

For analysis of BAL cells, the following antibodies were used: CD3 PE-Cy7, F4/80 FITC (eBioscience); CCR3 APC (R&D Systems) and Ly6G-PerCP Cy5.5 (BD Biosciences). Stained cells resuspended in PBS were acquired on a BD LSR II (BD Biosciences) and were analyzed on FlowJo software (Tree Star). To study lung, mediastinal lymph nodes (mLN) and spleen T-cell responses, these organs were removed and mechanically disrupted in RPMI 1640 media. Cells were passed through a 40 μm filter. Red blood cells from lung and spleen were lysed with a solution of Red Blood Cells lysis (Sigma Aldrich). Cells from mLN, lung and spleen were transferred to a 96-wells round-bottom plate and were stimulated for 5 hours with a mix containing phorbol 12-myristate 13-acetate (50 ng/ml) and ionomycin (500 ng/ml; Sigma-Aldrich) together. For cytokines detection, FcRs were blocked with mouse CD16/CD32 mAbs (eBioscience). The following antibodies were used for surface staining: CD3 PE-Cy7, CD103 PE, CD11c BV510, MHC II BV421, CD11b APC-H7 (eBioscience), CCR3 APC (R&D Systems), Ly6G PerCP-Cy5.5 (BD Biosciences) and F4/80 FITC (eBioscience) to analyze monocytes/macrophages and dendritic cells. Lineage cocktail FITC, CD45 PE, ST2 APC, DX5 PE-Cy7, CD127 BV421 and CD25 BV510 (eBioscience) to analyze lung ILC2, natural killer and natural killer T cells. CD3 PE-Cy7, CD4 BV421, CD25 BV510, (eBioscience), CD8 APC-H7 and CD44 FITC (BD Pharmingen) to analyze Treg cells. CD3 APC-Cy7, CD4 BV421 (eBioscience) and CD8 APC-H7 (BD Pharmingen) to analyze T cells. The cells were fixed and permeabilized using a Cytofix/Cytoperm kit (BD Biosciences) and stained intracellularly with anti-IL-4 APC clone 11B11, anti-IFNy PerCP-Cy5.5 clone XMG1.2 (BD Pharmingen) anti-IL-17A PE clone TC11-18H10.1 (Biolegend) to analyze T helper cells, cytokines production and Foxp3 PerCP-Cy5.5 (eBioscience) for Treg cells.

### Airway Hyperresponsiveness Measurement

Dynamic lung resistances were measured using a flexiVent (SCIREQ, Emka). Mice were anesthetized and connected via an endotracheal cannula to a flexiVent system. After initiating mechanical ventilation, the mouse was paralyzed with an intraperitoneal injection of 100 μl of a 10 mg/ml solution of rocuronium bromide and nebulized with methacholine (Sigma-Aldrich) (0-20 mg/ml). The animal was ventilated at a respiratory rate of 150 breaths/min and a tidal volume of 10 ml/kg against a positive end expiratory pressure of 3 cmH2O. Airway resistances wee continuously monitored and recorded according to manufacturer's instructions.

### Results

Inventors have shown that the respiratory function is improved in mice with the peptide Derp2.1 (FIG. 1B and 1C). They also evaluated inflammation environment in broncho-alveolar lavage (BAL): they have shown that neutrophils and eosinophils are reduced in BAL (FIG. 1D). Moreover, inventors have demonstrated that there is an increase of regulators lymphocytes T and a reduction of natural killer cells and of Derp2 specific IgE in the BAL (FIG. 1E, 1F and 1G).

Thus, the polypeptide derp2.1 is a new tool to treat the allergic asthma by a restoration of lung function and reduction of inflammatory environment.

### REFERENCES

Throughout this application, various references describe the state of the art to which this invention pertains. The disclosures of these references are hereby incorporated by reference into the present disclosure.

Chen KW, Fuchs G, Sonneck K, Gieras A, Swoboba I, Douladiris N, et al. Reduction of the in vivo allergenicity of Derp 2, the major house-dust mite allergen, by genetic engineering. Mol Immunol 2008; 45:2486-98.

1. A method of treating allergic asthma in a subject in need thereof comprising a step of administering to said subject a therapeutically effective amount of a polypeptide comprising a biologically active fragment of the house-dust mite Dermatophagoides pteronyssinus Der p 2.

2. The method according to claim 1, wherein the subject suffers or is susceptible to suffer from a severe asthma allergy.

3. The method according to claim 1, wherein the biologically active fragment of the house-dust mite Dermatophagoides pteronyssinus Der p 2 comprises:
a) the sequence consisting of amino acids 18 to 70 of SEQ ID NO: 1; or
b) a sequence having at least 80% identity with the sequence of (a); or
c) at least six consecutive amino acids of the sequence of (a) or (b).

4. The method according to claim 1, wherein, the polypeptide has a sequence consisting of amino acids 18 to 70 of SEQ ID NO: 1.

5. The method according to claim 1, wherein, the polypeptide is inserted in bacterium-like particles (BLP).

6. The method according to claim 1, wherein, administration of the polypeptide is performed by topical administration.

7. The method according to claim 6, wherein, the topical administration is performed by application of a patch.

8. The method according to claim 1, wherein administration of the polypeptide is performed by oral administration.

9. The method according to claim 8, wherein the oral administration of the polypeptide is performed by sublingual administration.

10. The method according to claim 1, wherein administration of the polypeptide is performed by subcutaneous administration.

11. The method according to claim 1, wherein the polypeptide is administered in combination with a classical treatment of allergic asthma.
